(19) United States
(12) Patent Application Publication
(10) Pub. No.: US 2025/0177271 A1
(43) Pub. Date: Jun. 5, 2025

(54) SCALP SUNSCREEN COMPOSITIONS AND METHODS OF USE

(71) Applicant: Steven Wang, Dana Point, CA (US)

(72) Inventor: Steven Wang, Dana Point, CA (US)

(21) Appl. No.: 18/525,400

(22) Filed: Nov. 30, 2023

(51) Int. Cl. A61K 8/40; A61K 8/35; A61K 8/37; A61K 8/64; A61K 8/65; A61K 8/81; A61K 8/92; A61K 8/9789; A61Q 5/06; A61Q 17/04; A61Q 19/00

(52) U.S. Cl. CPC A61K 8/40; A61K 8/35; A61K 8/37; A61K 8/64; A61K 8/65; A61K 8/8176; A61K 8/922; A61K 8/9789; A61Q 5/06; A61Q 17/04; A61Q 19/007; A61K 2800/522

(57) ABSTRACT

Compositions and methods of use are disclosed which can provide protection against ultraviolet radiation to a user's scalp and hair when applied thereto. Such compositions may further allow for the user to style their hair and aid in nourishment and strengthening of the user's hair fibers. The ingredients of these compositions can include UV filters, particularly homosalate, octisalate, avobenzone, and octocrylene, styling agents of polyvinylpyrrolidone and polyvinylpyrrolidone/vinyl acetate copolymer, and a plethora of skin conditioning agents derived from herb extracts, plant seeds, and amino acids. Numerous additives can be present in these compositions as well, ranging from solvents, antioxidants, pH adjusters, emulsion stabilizers, rheology modifiers, humectants, and preservatives.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

### 1. Field of Invention

The present application relates to sunscreen compositions and the application thereof to a user's skin and hair. More specifically, the present application relates to sunscreen compositions which may be applied to a user's scalp to protect against UV rays, style the user's hair, and nourish the user's hair.

### 2. Related Art

Solar ultraviolet (UV) radiation encompasses three sub-categories of rays of different wavelengths: long wave UVA (320-400 nm), medium wave UVB (280-320 nm), and short wave UVC (100-280 nm). While more than 99% of UVC is absorbed by the ozone layer, a significant portion of UVA and UVB reach Earth's surface and consequently affects the biological and pathological processes of humans, especially within their skin and hair. UVA penetrates into the dermis, while UVB is almost completely absorbed by the epidermis. Though the impact of UV radiation on the skin stimulates the production of vitamin D, an important nutrient for tissue homeostasis, the combination of UVA and UVB also triggers negative processes which can result in erythema, edema, sunburn, photoaging, skin cancer, and hair damage.

In recent years, the detrimental effects of UV radiation from the sun on the human body have been extensively studied, revealing a plethora of adverse consequences, particularly on the delicate skin of the scalp and the hair. UV radiation poses a significant threat, with skin cancer and sunburns being among the most alarming outcomes. Statistics indicate a staggering 5 million skin cancer cases diagnosed annually in the USA, with about 15% of these malignancies found on the head, emphasizing the critical need for effective protection in this area.

Beyond the risks of skin cancer and sunburn, UV radiation has been identified as a leading factor in several hair-related issues. Premature greying of hair, a phenomenon experienced by a considerable portion of the population, has been linked to prolonged exposure to UV rays. Moreover, UV ray exposure damages the hair fibers, causing them to become brittle and dry. This degradation not only weakens the hair but also robs it of its natural luster and strength. Additionally, the vibrant colors that consumers cherish in their hair are notably susceptible to fading due to UV radiation exposure, diminishing the overall aesthetic appeal. Furthermore, emerging research suggests a potential link between UV radiation and hair loss, and studies have shown that increased hair loss is reported in patients exposed to repeated episodes of sunburn in the scalp skin.

As alarming as these consequences are, a substantial barrier inhibits effective protection: the reluctance of consumers to use conventional sunscreens on their scalp. It is easy to understand this reluctance, as existing sunscreen products are often greasy, oily, and heavy, rendering them unsuitable for application on the head. These products not only weigh down the hair but also flatten hairstyles, causing dissatisfaction among users. Additionally, the lack of established habits in applying sunscreen to the scalp further exacerbates the problem.

In light of these challenges, there exists a pressing need for an innovative solution that effectively shields the scalp and hair from the harmful effects of UV radiation while being convenient and comfortable to apply.

### BRIEF SUMMARY

To solve these and other problems, compositions, and methods of using the same are disclosed which may provide one or more of the following: protection against ultraviolet (UV) rays to a user's scalp and/or hair, the capability to style the user's hair, and nourishment of the user's hair fibers. These compositions may yield these results in part by the ingredients present in the composition; UV filters may at least partially block out UV rays, styling agents may better allow the user to shape and form their hair and skin conditioning agents could aid in strengthening of the hair's health. Additives may be included in the composition, such as a solvent, pH adjusters, emulsion stabilizers, rheology modifiers, preservatives, humectants and antioxidants, to assist in secondary functions, such as the storage of the composition, transportation of the composition, and more.

The UV filters of a composition can include homosalate at a concentration of 1 to 15%, octisalate at a concentration of 1% to 5% of the composition, avobenzone at a concentration of 0.1% to 3%, and octocrylene at a concentration of 1% to 10%, and ideally the concentrations of these ingredients will be at around 6.375%, 3%, 1.8%, and 6.224%, respectively.

Styling agents include polyvinylpyrrolidone at a concentration of 0.1% to 5% of the composition, ideally at 2.5%, and polyvinylpyrrolidone/vinyl acetate copolymer at a concentration of 0.1% to 5% of the composition, ideally at around 2.5%. Lift and volume can be added to the user's hair by these ingredients, allowing for the user to style their hair more effectively.

A variety of skin conditioning agents can be included in these compositions. Herb extracts, such as Glycyrrhiza glabra root extract, leuconostoc/radish root ferment filtrate, Camellia sinensis leaf extract, Scenedesmus rubescens extract, and Coffea arabica are some examples, each of which can independently be present at a concentration range of 0.1% to 3% but ideally at around 0.5%. Other types of agents from plants include Camellia sinensis seed oil and Carthamus tinctorius seed oil, both of which can independently be present at a concentration range of 0.1% to 5%, preferably around 1.5%. Another type of suitable skin conditioning agents includes amino acids, particularly collogen amino acid and silk amino acid, both of which can independently be present at a concentration range of 0.1% to 5%, ideally at around 1.5%.

An emulsion stabilizer, such as carbomer, can be present at a concentration of 0.01% to 1%, ideally at around 0.5%. Suitable rheology modifiers and concentrations thereof within these compositions are hydroxyethyl acrylate/sodium acryloyldimethyl taurate copolymer at 0.1% to 1%, ideally at around 0.75% and xanthum gum at 0.01% to 1%, ideally at around 0.5%. The combination of the following humectants has been found to be effective at the following concentrations: glycerin at 0.1% to 2% (ideally at around 0.5%), butylene glycol at 0.5% to 4% (ideally at around 1%), and sorbitol at 0.5% to 5% (ideally at around 2.5%). Preservatives fit for these compositions include benzyl alcohol, which can be present at a concentration of 0.01% to 2% of the composition (ideally at around 0.5%), and phenoxyethanol, which can be present at a concentration of 0.1% to 2% (ideally at around 0.5%). Niacinamide can be used as the antioxidant at a concentration of 0.1% to 2% of the composition, ideally at around 1%, and aminomethyl propanol may be used as the pH adjuster at a concentration of 0.1% to 1.5% of the composition, ideally at around 0.5%.

Any composition can be formulated as a solution with a solvent such as water. A user may apply these compositions to a region of or the entirety of their hair and/or scalp, such as by pouring the composition thereon or pouring the composition onto their hands and thereafter lathering their scalp and hair with their hands.

All of these embodiments are contemplated to be within the scope of this disclosure. These and other embodiments will become readily apparent to those skilled in the art from the following detailed description of the preferred embodiments, the invention not being limited to any particular preferred embodiments disclosed.

### BRIEF DESCRIPTION OF THE DRAWING

These and other features and advantages of the various embodiments disclosed herein will be better understood with respect to the following description and drawing, in which:

FIG. 1 shows an example of the styling capabilities of an exemplary composition according to this disclosure.

### DETAILED DESCRIPTION

The present description encompasses various embodiments of compositions and methods of use which, when applied to a user's skin, hair, or both can provide ultraviolet (UV) protection, hair styling capabilities, and/or hair nourishment. Such compositions can include a variety of ingredients, including UV filters, styling agents, and/or skin conditioning agents which can at least partially contribute to one or more of these beneficial properties. Additives can be included in these compositions, which may include solvents, emulsion stabilizers, rheology modifiers, humectants, preservatives, and pH adjusters, each of which can further contribute to those beneficial properties and/or serve secondary functions, such as making the composition easier to store, handle, use, etc. A user may apply this composition to their hair and/or scalp, similar to how one could apply a conventional hair gel product, which may result in the user's hair and/or scalp being protected from UV rays, the user being more capable of styling their hair, and/or a hair subsistence effect.

Each of the ingredients which can be present within these compositions will be quantified in this disclosure as a percentage of the total concentration of the composition. Ranges in which these ingredients may be present, alongside narrower, preferred ranges, will be disclosed alongside the concentration of those ingredients within an exemplary composition which has been shown to give effectual results. Specific ingredients suitable for use in these compositions will be highlighted, but it is to be understood that alternatives may be used alongside or in place of any particular ingredient, as would be appreciated by those skilled in the art. For example, while only one pH adjuster ingredient, aminomethyl propanol, is discussed below, it is to be understood that there are other pH adjusters which can substitute or be present alongside aminomethyl propanol in a composition.

UV ray protection may be provided at least partially by UV filters present in a composition. Particular UV filters may include homosalate, octisalate, avobenzone, and octocrylene. Homosalate can be present in the composition at a concentration range of 1% to 15%, preferably 4% to 8%, and most preferably 6% to 7%. As for Octisalate, it may be present in the composition at a concentration range of 1% to 5%, preferably 2% to 4%, and most preferably 2.5% to 3.5%. The UV filter of avobenzone can be present in the composition at a concentration range of 0.1% to 3%, preferably 0.5% to 2.25%, and most preferably 1% to 2%. Octocrylene may be present in the composition at a composition range of 1% to 10%, preferably 4% to 8%, and most preferably 6% to 7%. Other UV filters that can be used and their potential concentration ranges include, but are not limited to, zinc oxide at a concentration range of 1% to 20% and titanium dioxide at a concentration range of 1% to 20%. The exemplary composition, which achieved a sunscreen protection factor (SPF) of 40, had the following concentrations of these UV filters: 6.375% homosalate, 3% octisalate, 1.8% avobenzone, and 6.224% octocrylene. Compositions may vary in their Sun Protection Factor (SPF) based on the composition's ingredients and the concentrations thereof; the SPF value of the compositions contemplated herein may range from an SPF of 15 to an SPF of 60; the critical wavelength (CW) value of the compositions contemplated herein may range from a CW 370 nm to a CW of 400 nm. Higher concentrations values of these UV filters have been found to result in increased SPF and CW while sacrificing a preferred texture and feel of the composition, while lower concentrations were found to have the inverse effect. Too high of a concentration of UV filters can decrease the holding ability, the styling capabilities, and lifting capability of the product. Too low of a concentration of UV filters can lead to overly strong holding capabilities of the final product, making the hair strands stick together. The exemplary composition is believed to strike the best balance between these two factors, providing exceptional protection against UV rays while being pleasant to apply and remain upon a user's scalp and hair. At the same time, the exemplary composition, having a combination of styling agents as discussed below, can create an ideal result of lifting, styling, and adding volume to the hair.

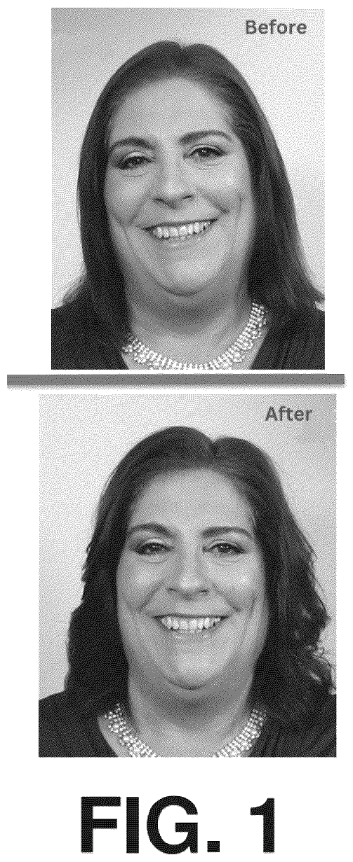

The styling agents of a composition may aid in allowing a user to style their hair via lifting, and/or adding volume to the user's hair. Such styling agents can include polyvinylpyrrolidone and/or polyvinylpyrrolidone/vinyl acetate (VP/VA) copolymer. Any styling agent can independently be present in the composition at a concentration range of 0.1% to 5%, preferably 0.5% to 4%, and most preferably 1.5% to 3%. The exemplary composition had polyvinylpyrrolidone present at a concentration of 2.5% and VP/VA copolymer present at a concentration of 2.5%; an example of how this exemplary composition has been used to style the hair of a user via a before and after comparison is shown in FIG. 1. While this combination of styling agents has proven to be effective in these compositions, it is contemplated that various film-forming agents used in conventional hair styling products, such as acrylates, acrylamides, methacrylates, and copolymers, can be used alongside or in place of any styling agent present in these compositions. Some specific examples include, but are not strictly limited to, vinylpyrrolidone/alkene copolymers like VP/eicosene copolymer or VP/hexadecene copolymer, PVM/MA copolymer, polyalkylsilsesquioxanes, polyacrylamide, hydroxymethylcellulose, hydroxyethylcellulose, and hydroxypropylcellulose.

Skin conditioning agents of a composition may contribute to the composition's capability to nourish and strengthen hair fibers. These skin conditioning agents may be derived from plants and/or amino acids. Specific examples which can originate from herb extracts include Glycyrrhiza glabra (licorice) root extract, leuconostoc/radish root ferment filtrate, Camellia sinensis leaf extract, Scenedesmus rubescens extract, and Coffea arabica (coffee) extract. All these extracts may be present in the composition at a concentration of 0.1% to 3%, preferably 0.25% to 2%, and most preferably 0.5% to 1.5%. Camellia sinensis seed oil and Carthamus tinctorius (safflower) seed oil are two examples of skin conditioning agents which can be derived from plant seeds. Both may be present in the composition at a concentration range of 0.1% to 5%, preferably 0.5% to 3%, and most preferably 1% to 2%. As for amino acids, collogen amino acid and silk amino acid are suitable skin conditioning agents. Collogen amino acid and silk amino acid may both independently be present in the composition at a concentration range of 0.1% to 5%, preferably 0.5% to 3%, and most preferably 1% to 2%. The exemplary composition had Glycyrrhiza glabra (licorice) root extract at a concentration of 0.5%, leuconostoc/radish root ferment filtrate at a concentration of 0.5%, Camellia sinensis leaf extract at a concentration of 0.5%, Scenedesmus rubescens extract at a concentration of 0.5%, Coffea arabica (coffee) extract at a concentration of 0.5%, Camellia sinensis seed oil at a concentration of 1.5%, Carthamus tinctorius (safflower) seed oil at a concentration of 1.5%, collagen amino acid at a concentration of 1.5%, and silk amino acid at a concentration of 1.5%.

Additives may be present in a composition alongside any combination of the UV filters, skin conditioning agents, and/or styling agents. These additives may further contribute to the properties of UV filtration, styling, and/or hair proliferation (e.g., an additive may partially contribute to the degree of UV filtration of a composition), but they could also or alternatively aid in the practical usage of the composition, stability of the composition, etc., as would be appreciated by those skilled in the art. The additives of a composition can include one or more of the following: emulsion stabilizer, rheology modifier, humectant, preservative, antioxidant, pH adjuster, and solvent.

The emulsion stabilizer can include, but is not strictly limited to, carbomer. The total concentration of the emulsion stabilizer present in the composition can range from 0.01% to 1%, preferably 0.05% to 0.75%, and most preferably 0.1% to 0.5%. Carbomer was present at a concentration of 0.5% in the exemplary composition.

Suitable rheology modifiers which may be present in a composition include hydroxyethyl acrylate/sodium acryloyldimethyl taurate copolymer and/or xanthum gum. The former may be present at a concentration range of 0.01% to 1%, preferably 0.2% to 0.9%, and most preferably 0.3% to 0.8%, while the latter may be present at a concentration range of 0.01% to 1%, preferably 0.05% to 0.9%, and most preferably 0.1% to 0.8%. The exemplary composition had hydroxyethyl acrylate/sodium acryloyldimethyl taurate copolymer present at a concentration of 0.75% and xanthum gum present at a concentration of 0.5%.

Among the humectants fit to for the compositions of this disclosure include glycerin, butylene glycol, and/or sorbitol. Glycerin can be present at a concentration range of 0.1% to 2%, preferably 0.2% to 1%, and most preferably 0.3% to 0.7%. Butylene glycol may be present at higher concentration ranges of 0.5% to 4%, preferably 0.75% to 3%, and most preferably 1% to 2%. Similarly, sorbitol can be present at a concentration range of 0.5% to 5%, preferably 1% to 3%, and most preferably 1.5% to 2.5%. The exemplary composition had each of these three humectants present at the following concentrations: glycerin at 0.5%, butylene glycol at 1%, and sorbitol at 2.5%.

The preservatives which have been found to be effective in the compositions disclosed herein include benzyl alcohol and/or phenoxyethanol. Benzyl alcohol can be present at a concentration range of 0.01% to 2%, preferably 0.1% to 0.75%, and most preferably 0.2% to 0.5%. Compositions may have phenoxyethanol present at a concentration range of 0.1% to 2%, preferably 0.3% to 1%, and most preferably 0.4% to 0.6%. The exemplary composition had both preservatives present at the following concentrations: benzyl alcohol at 0.5% and phenoxyethanol at 0.5%.

Niacinamide can be used as an antioxidant in the composition, which may particularly protect against skin cancer. Whichever antioxidant or combination of antioxidants is used, the total concentration of antioxidant can range from 0.1% to 2%, preferably 0.3% to 1.5%, and most preferably 0.7% to 1.25%. The exemplary composition had just niacinamide present at a concentration of 1%.

Aminomethyl propanol has been shown to be an effective pH adjuster for these compositions. The pH adjuster can be present at a concentration range of 0.1% to 2%, preferably 0.2% to 1%, and most preferably 0.3% to 0.7%. Aminomethyl propanol was present at a concentration of 0.5% in the exemplary composition.

The composition may include a solvent, ideally water, to carry the above ingredients and allow for easier transportation and application of the solvent. The concentration of solvent in the composition may make up the majority of a composition's concentration, in that the solvent can comprise at least 50% of the composition. In ideal compositions, the concentration of solvent can range from 50-85%, preferably 55%-80%, and most preferably 60-75%. Water was present at a concentration of about 63% in the exemplary composition.

These compositions can be made via mixture of the above ingredients, which could further involve measurement of the amounts of ingredients being mixed to arrive at certain concentration values of those ingredients within the composition. Individual ingredients or mixtures of ingredients can be mixed consecutively or sequentially; certain mixtures can thus be made which comprise some of the ingredients of the target composition before they are mixed with other ingredients and/or other mixtures.

A user may apply a composition via conventional methodologies, such as pouring the composition onto their scalp or pouring the composition onto their hands and thereafter lathering their scalp and hair with their hands, with further styling of their hair if needed. A portion of a user's scalp/hair or the entirety thereof may receive the composition during application thereof. A composition can be applied generally evenly throughout the user's hair, or a user may intentionally apply more of the composition to certain regions of their scalp/hair in case, for instance, those regions require more of the composition in order to be styled in a particular way or it is anticipated that region will have more exposure to UV rays relative to other regions.

The above description is given by way of example, and not limitation. Given the above disclosure, one skilled in the art could devise variations that are within the scope and spirit of this description. Further, the various features of the embodiments disclosed herein can be used alone, or in varying combinations with each other and are not intended to be limited to the specific combination described herein. Thus, the scope of the claims is not to be limited by the illustrated embodiments. Additional modifications and improvements of the present disclosure may also be apparent to those of ordinary skill in the art. Thus, the particular combination of parts and steps described and illustrated herein is intended to represent only certain embodiments of the present disclosure and is not intended to serve as limitations of alternative compositions and methods within the spirit and scope of the disclosure.

What is claimed is:

1. A composition for protecting a user's scalp and hair against UV radiation and for styling the user's hair, the composition comprising:
a UV filter, the UV filter comprising:
homosalate at a concentration of 1% to 15% of the composition;
octisalate at a concentration of 1% to 5% of the composition;
avobenzone at a concentration of 0.1% to 3% of the composition; and
octocrylene at a concentration of 1% to 10% of the composition;
a styling agent, the styling agent comprising:
polyvinylpyrrolidone at a concentration of 0.1 to 5% of the composition; and
polyvinylpyrrolidone/vinyl acetate copolymer at a concentration of 0.1 to 5% of the composition.

2. The composition of claim 1, wherein the composition further comprises:
a skin conditioning agent for nourishing the user's hair, the skin conditioning agent comprising:
Glycyrrhiza glabra root extract at a concentration of 0.1% to 3% of the composition;
leuconostoc/radish root ferment filtrate at a concentration of 0.1% to 3% of the composition;
Camellia sinensis leaf extract at a concentration of 0.1% to 3% of the composition;
Scenedesmus rubescens extract at a concentration of 0.1% to 3% of the composition; and
Coffea arabica extract at a concentration of 0.1% to 3% of the composition.

3. The composition of claim 1, wherein the composition further comprises:
a skin conditioning agent for nourishing the user's hair, the skin conditioning agent comprising:
Camellia sinensis seed oil at a concentration of 0.1% to 3% of the composition; and
Carthamus tinctorius seed oil at a concentration of 0.1% to 3% of the composition.

4. The composition of claim 1, wherein the composition further comprises:
a skin conditioning agent for nourishing the user's hair, the skin conditioning agent comprising:
collogen amino acid at a concentration of 0.1% to 5% of the composition; and
silk amino acid at a concentration of 0.1% to 5% of the composition.

5. The composition of claim 1, wherein the composition further comprises:
an emulsion stabilizer at a concentration of 0.01% to 1% of the composition, the emulsion stabilizer comprising carbomer.

6. The composition of claim 1, wherein the composition further comprises:
a rheology modifier, the rheology modifier comprising:
hydroxyethyl acrylate/sodium acryloyldimethyl taurate copolymer at a concentration of 0.01% to 1% of the composition; and
xanthum gum at a concentration of 0.01% to 0.5% of the composition.

7. The composition of claim 1, wherein the composition further comprises:
a humectant, the humectant comprising:
glycerin at a concentration of 0.1% to 2% of the composition;
butylene glycol at a concentration of 0.5% to 4% of the composition; and
sorbitol at a concentration range of 0.5% to 5% of the composition.

8. The composition of claim 1, wherein the composition further comprises:
a preservative, the preservative comprising:
benzyl alcohol at a concentration of 0.01% to 2% of the composition; and
phenoxyethanol at a concentration of 0.1% to 2% of the composition.

9. The composition of claim 1, wherein the composition further comprises:
an antioxidant at a concentration of 0.1% to 2% of the composition, the antioxidant comprising niacinamide.

10. The composition of claim 1, wherein the composition further comprises:
a pH adjuster at a concentration of 0.1% to 2% of the composition, the pH adjuster comprising aminomethyl propanol.

11. A method of protecting a scalp and hair of a user from UV radiation and styling the hair of the user with a composition, the method comprising the step of:
applying the composition to the scalp and hair;
wherein the composition comprises:
a UV filter, the UV filter comprising:
homosalate at a concentration of 1% to 15% of the composition;
octisalate at a concentration of 1% to 5% of the composition;
avobenzone at a concentration of 0.1% to 3% of the composition; and
octocrylene at a concentration of 1% to 10% of the composition;
a styling agent, the styling agent comprising:
polyvinylpyrrolidone at a concentration of 0.1% to 5% of the composition; and
polyvinylpyrrolidone/vinyl acetate copolymer at a concentration of 0.1 to 5% of the composition.

12. The method of claim 11, wherein the composition further comprises:
a skin conditioning agent for nourishing the user's hair, the skin conditioning agent comprising:
Glycyrrhiza glabra root extract at a concentration of 0.1% to 3% of the composition;
leuconostoc/radish root ferment filtrate at a concentration of 0.1% to 3% of the composition;
Camellia sinensis leaf extract at a concentration of 0.1% to 3% of the composition;
Scenedesmus rubescens extract at a concentration of 0.1% to 3% of the composition; and
Coffea arabica extract at a concentration of 0.1% to 3% of the composition.

13. The composition of claim 11, wherein the composition further comprises:
a skin conditioning agent for nourishing the user's hair, the skin conditioning agent comprising:
Camellia sinensis seed oil at a concentration of 0.1% to 5% of the composition; and
Carthamus tinctorius seed oil at a concentration of 0.1% to 5% of the composition.

14. The method of claim 11, wherein the composition further comprises:
a skin conditioning agent for nourishing the user's hair, the skin conditioning agent comprising:
collogen amino acid at a concentration of 0.1% to 5% of the composition; and
silk amino acid at a concentration of 0.1% to 5% of the composition.

15. The method of claim 11, wherein the composition further comprises:
an emulsion stabilizer at a concentration of 0.01% to 1% of the composition, the emulsion stabilizer comprising carbomer.

16. The method of claim 11, wherein the composition further comprises:
a rheology modifier, the rheology modifier comprising:
hydroxyethyl acrylate/sodium acryloyldimethyl taurate copolymer at a concentration of 0.01% to 1% of the composition; and
xanthum gum at a concentration of 0.01% to 1% of the composition.

17. The method of claim 11, wherein the composition further comprises:
a humectant, the humectant comprising:
glycerin at a concentration of 0.1% to 2% of the composition;
butylene glycol at a concentration of 0.5% to 4% of the composition; and
sorbitol at a concentration range of 0.5% to 5% of the composition.

18. The method of claim 11, wherein the composition further comprises:
a preservative, the preservative comprising:
benzyl alcohol at a concentration of 0.01% to 2% of the composition; and
phenoxyethanol at a concentration of 0.1% to 2% of the composition.

19. The method of claim 11, wherein the composition further comprises:
an antioxidant at a concentration of 0.1% to 2% of the composition, the antioxidant comprising niacinamide.

20. The method of claim 11, wherein the composition further comprises:
a pH adjuster at a concentration of 0.1% to 2% of the composition, the pH adjuster comprising aminomethyl propanol.

21. The method of claim 11, wherein the UV filter is operative to prevent premature greying of the user's hair.
